(19) United States
(12) Patent Application Publication
(10) Pub. No.: US 2005/0129685 A1
(43) Pub. Date: Jun. 16, 2005

(54) Use of IL-1 blockers to prevent corneal inflammation and neovascularization

(76) Inventors: Jingtai Cao, Chappagua, NY (US); Stanely J. Wiegand, Croton on Hudson, NY (US)

Correspondence Address: REGENERON PHARMACEUTICALS, INC, 777 OLD SAW MILL RIVER ROAD, TARRYTOWN, NY, 10591, US

(21) Appl. No.: 10/945,068

(22) Filed: Sep. 20, 2004

Related U.S. Application Data
(60) Provisional application No. 60/503,854, filed on Sep. 18, 2003.

(51) Int. Cl. A61K 48/00; A61K 39/395; A61K 38/17

(52) U.S. Cl. 424/143.1; 514/44; 424/145.1; 514/12

(57) ABSTRACT

Methods of preventing, reducing, or treating corneal inflammation and neovascularization in a subject in need thereof comprising administering IL-1 blockers are provided. More specifically, the methods comprise administering IL-1 receptor-based blockers to prevent, reduce or treat corneal inflammation and neovascularization.

### CROSS-REFERENCE TO RELATED APPLICATIONS

This application claims the benefit under 35 USC § 119(e) of U.S. Provisional 60/503,854 filed 18 Sep. 2003, which application is herein specifically incorporated by reference in its entirety.

### BACKGROUND

1. Field of the Invention

The invention relates to methods of using interleukin-1 (IL-1) blockers or antagonists to reduce, prevent, or treat corneal inflammation and neovascularization associated with corneal injury, in particular those associated with corneal transplant surgery.

2. Description of Related Art

It has previously been reported that inhibition of interleukin-1 (IL-1) with IL-1 Ra, a natural IL-1 antagonist, suppresses neovascularization in rat models of corneal angiogenesis and adjuvant arthritis (Coxon et al. (2002) Arthritis and Rheumatism 46(10):2604-2612).

### BRIEF SUMMARY OF THE INVENTION

The invention is based, in part, on the finding that administration of a receptor-based blocker or antagonist of an interleukin-1 (IL-1)-mediated activity prevents corneal inflammation and neovascularization in an animal model of corneal injury.

In a first aspect, of the invention features a method of treating, inhibiting or reducing corneal inflammation and neovascularization in a subject in need of or at risk thereof, comprising administering an interleukin-1 (IL-1) antagonist, such that corneal inflammation and/or neovascularization is treated, inhibited, or reduced.

An IL-1 blocker or antagonist is a compound capable of blocking or inhibiting the biological action of IL-1, including fusion proteins capable of trapping IL-1, such as an IL-1 trap, interleukin-1 antagonist (IL-1 ra), an anti-IL-1 antibody or fragment thereof, an anti-IL-1 receptor antibody or fragment thereof, a small molecule, or a nucleic acid capable of interfering with the expression of IL-1.

In a preferred embodiment, the IL-1 antagonist is an IL-1-specific fusion protein comprising two IL-1 receptor components and a multimerizing component, for example, an IL-1 trap described in U.S. patent publication No. 2003/0143697, published 31 Jul. 2003, herein specifically incorporated by reference in its entirety. In a specific embodiment, the IL-1 trap is the fusion protein shown in SEQ ID NO:2, 4, 6, 8, 10, 12, 14, 16, 18, 20, 22, 24, 26. A preferred IL-1 trap is shown in SEQ ID NO:10. In another embodiment, the IL-1 antagonist is an antibody or antibody fragment capable of binding IL-1a and/or IL-1β. In another embodiment, the IL-1 antagonist is an anti-IL-1 receptor (IL-1 R1 or IL-1 RAcp), or a fragment thereof. In specific embodiments, the IL-1 antagonist is a modified IL-1 trap comprising one or more receptor components and one or more immunoglobulin-derived components specific for IL-1 and/or an IL-1 receptor. In another embodiment, the IL-1 antagonist is a modified IL-1 trap comprising one or more immunoglobulin-derived components specific for IL-1 and/or an IL-1 receptor. In another embodiment, the IL-1 antagonist is IL-1α (SEQ ID NO:27 (full-length molecule); SEQ ID NO:28 (mature protein). In yet another embodiment, the IL-1 antagonist is a nucleic acid capable of interfering with the expression of IL-1. Examples of IL-1 antagonist nucleic acids include, for example, antisense molecules, inhibitory ribozymes designed to catalytically cleave gene mRNA transcripts encoding IL-1α, IL-1β, IL-1 R1, IL-1RAcp, or short interfering RNA (siRNA) molecules.

The subject treated by the method of the invention is preferably a human subject in need of or at risk for development of corneal inflammation and neovascularization is a human subject. In one embodiment, the subject is a patient who has undergone corneal transplant surgery.

The method of the invention includes administration of the IL-1 blocker or antagonist by any means known to the art, for example, subcutaneous, intramuscular, intranasal, intraarterial, intravenous, topical, transvaginal, transdermal, transanal administration or oral routes of administration. In one embodiment, administration is topical to the eye or subconjunctival administration.

In a second related aspect, the invention features a method of reducing the incidence of corneal inflammation and neovascularization in a subject in need or at risk thereof, comprising administering to the subject an IL-1 blocker or antagonist such that the incidence of corneal inflammation and neovascularization is reduced. Such corneal inflammation and neovascularization can result from corneal injury (e.g. physical trauma, foreign body) and corneal surgery, including corneal transplant surgery.

In a third aspect, the invention features a pharmaceutical composition comprising an IL-1 trap in a pharmaceutically acceptable carrier. Such pharmaceutical compositions may be liquid, gel, ointment, salve, slow release formulations or other formulations suitable for ophthalmic administration.

Other objects and advantages will become apparent from a review of the ensuing detailed description.

### DETAILED DESCRIPTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

As used in this specification and the appended claims, the singular forms “a”, “an”, and “the” include plural references unless the context clearly dictates otherwise. Thus for example, a reference to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference.

General Description

The corneal injury animal model directly mimics clinical conditions which are associated with corneal neovascularization, such as physical injury, foreign body response, corneal transplantation, etc., and is thus distinct from conditions where angiogenesis is artificially provoked by application of a single, defined exogenous angiogenic factor (such as bFGF or VEGF). As shown below, blocking IL-1-mediated neovascularization is useful and effective in the treatment of inflammatory neovascularization as it produces fewer of the adverse effects associated with current treatments, such as systemic administration of anti-inflammatory/angiostatic steroids.

Definitions

The terms “treatment”, “treating”, and the like are used herein to generally mean obtaining a desired pharmacologic and/or physiologic effect. The effect may be prophylactic in terms of completely or partially preventing a disease, condition, or symptoms thereof, and/or may be therapeutic in terms of a partial or complete cure for a disease or condition and/or adverse effect attributable to the disease or condition.

“Treatment” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition, i.e., arresting its development; or (c) relieving the disease or condition, i.e., causing regression of the disease or condition. The population of subjects treated by the method of the disease includes subject suffering from the undesirable condition or disease, as well as subjects at risk for development of the condition or disease.

By the term “therapeutically effective dose” is meant a dose that produces the desired effect for which it is administered. The exact dose will depend on the purpose of the treatment, and will be ascertainable by one skilled in the art using known techniques (see, for example, Lloyd (1999) The Art, Science and Technology of Pharmaceutical Compounding).

The terms “blocker,” “antagonist,” or “inhibitor” are used interchangeably to mean a substance that retards or prevents a chemical or physiological reaction or response. Common blockers or inhibitors include, but are not limited to, antisense molecules, antibodies, antagonists and their derivatives. More specifically, an example of an IL-1 blocker or inhibitor is an IL-1 receptor-based antagonist including, but not limited to, IL-1R1-IL-1AcP-FcΔC1 (a).

IL-1 Trap Antagonists

Interleukin-1 (IL-1) traps are multimers of fusion proteins containing IL-1 receptor components and a multimerizing component capable of interacting with the multimerizing component present in another fusion protein to form a higher order structure, such as a dimer. Cytokine traps are a novel extension of the receptor-Fc fusion concept in that they include two distinct receptor components that bind a single cytokine, resulting in the generation of antagonists with dramatically increased affinity over that offered by single component reagents. In fact, the cytokine traps that are described herein are among the most potent cytokine blockers ever described. Briefly, the cytokine traps called IL-1 traps are comprised of the extracellular domain of human IL-1 R Type I (IL-1 RI) or Type II (IL-1RII) followed by the extracellular domain of human IL-1 Accessory protein (IL-1AcP), followed by a multimerizing component. In a preferred embodiment, the multimerizing component is an immunoglobulin-derived domain, such as, for example, the Fc region of human IgG, including part of the hinge region, the CH2 and CH3 domains. Alternatively, the IL-1 traps are comprised of the extracellular domain of human IL-1AcP, followed by the extracellular domain of human IL-1RI or IL-1 RII, followed by a multimerizing component. For a more detailed description of the IL-1 traps, see WO 00/18932, which publication is herein specifically incorporated by reference in its entirety. Preferred IL-1 traps have the amino acid sequence shown in SEQ ID NOs: 2, 4, 6, 8, 10, 12, 14, 16, 18, 20, 22, 24, and 26.

In specific embodiments, the IL-1 antagonist comprises an antibody fragment capable of binding IL-1α, IL-1β, IL-1 R1 and/or IL-1RAcp, or a fragment thereof. One embodiment of an IL-1 antagonist comprising one or more antibody fragments, for example, single chain Fv (scFv), is described in U.S. Pat. No. 6,472,179, which publication is herein specifically incorporated by reference in its entirety. In all of the IL-1 antagonist embodiments comprising one or more antibody-derived components specific for IL-1 or an IL-1 receptor, the components may be arranged in a variety of configurations, e.g., a IL-1 receptor component(s)-scFv(s)-multimerizing component; IL-1 receptor component(s)-multimerizing component-scFv(s); scFv(s)-IL-1 receptor component(s)-multimerizing component, etc., so long as the molecule or multimer is capable of inhibiting the biological activity of IL-1. In another embodiment, the IL-1 antagonist is IL-1ra, including the full length protein of SEQ ID NO:27 or the mature protein of SEQ ID NO:28.

Antisense Molecules

In one aspect of the invention, IL-1-mediated activity is blocked or inhibited by the use of IL-1 antisense nucleic acids. The present invention provides the therapeutic or prophylactic use of nucleic acids comprising at least six nucleotides that are antisense to a gene or cDNA encoding IL-1 or a portion thereof. As used herein, an IL-1 “antisense” nucleic acid refers to a nucleic acid capable of hybridizing by virtue of some sequence complementarity to a portion of an RNA (preferably mRNA) encoding IL-1. The antisense nucleic acid may be complementary to a coding and/or noncoding region of an mRNA encoding IL-1. Such antisense nucleic acids have utility as compounds that prevent IL-1 expression, and can be used in the treatment of corneal transplant rejection. The antisense nucleic acids of the invention are double-stranded or single-stranded oligonucleotides, RNA or DNA or a modification or derivative thereof, and can be directly administered to a cell or produced intracellularly by transcription of exogenous, introduced sequences.

The invention further provides pharmaceutical compositions comprising a therapeutically effective amount of IL-1 antisense nucleic acid, and a pharmaceutically acceptable carrier, vehicle or diluent. The IL-1 antisense nucleic acids are of at least six nucleotides and are preferably oligonucleotides ranging from 6 to about 50 oligonucleotides. In specific aspects, the oligonucleotide is at least 10 nucleotides, at least 15 nucleotides, at least 100 nucleotides, or at least 200 nucleotides. The oligonucleotides can be DNA or RNA or chimeric mixtures or derivatives or modified versions thereof and can be single-stranded or double-stranded. In addition, the antisense molecules may be polymers that are nucleic acid mimics, such as PNA, morpholino oligos, and LNA. Other types of antisense molecules include short double-stranded RNAs, known as siRNAs, and short hairpin RNAs, and long dsRNA (>50 bp but usually ≧500 bp).

Short Interfering RNAs

In another embodiment, IL-1-mediated activity is blocked by blocking IL-1 expression. One method for inhibiting IL-1 expression is the use of short interfering RNA (siRNA) through RNA interference (RNAi) or post-transcriptional gene silencing (PTGS) (see, for example, Ketting et al. (2001) Genes Develop. 15:2654-2659). siRNA molecules can target homologous mRNA molecules for destruction by cleaving the mRNA molecule within the region spanned by the siRNA molecule. Accordingly, siRNAs capable of targeting and cleaving homologous IL-1 mRNA are useful for treating, reducing or preventing corneal transplant rejection.

Inhibitory Ribozymes

In another aspect of the invention, corneal transplant rejection may be treated, reduced or prevented by decreasing the level of IL-1 activity by using ribozyme molecules designed to catalytically cleave gene mRNA transcripts encoding IL-1, preventing translation of target gene mRNA and, therefore, expression of the gene product. Ribozymes are enzymatic RNA molecules capable of catalyzing the specific cleavage of RNA. The mechanism of ribozyme action involves sequence-specific hybridization of the ribozyme molecule to complementary target RNA, followed by an endonucleolytic cleavage event. The composition of ribozyme molecules must include one or more sequences complementary to the target gene mRNA, and must include the well known catalytic sequence responsible for mRNA cleavage. For this sequence, see, e.g., U.S. Pat. No. 5,093,246. While ribozymes that cleave mRNA at site-specific recognition sequences can be used to destroy mRNAs encoding IL-1, the use of hammerhead ribozymes is preferred. Hammerhead ribozymes cleave mRNAs at locations dictated by flanking regions that form complementary base pairs with the target mRNA. The sole requirement is that the target mRNA has the following sequence of two bases: 5′-UG-3′. The construction and production of hammerhead ribozymes is well known in the art. The ribozymes of the present invention also include RNA endoribonucleases (hereinafter “Cech-type ribozymes”) such as the one that occurs naturally in Tetrahymena thermophila (known as the IVS, or L-19 IVS RNA). The Cech-type ribozymes have an eight base pair active site that hybridizes to a target RNA sequence where after cleavage of the target RNA takes place. The invention encompasses those Cech-type ribozymes that target eight base-pair active site sequences that are present in the gene encoding IL-1.

Anti-IL-1 Human Antibodies and Antibody Fragments

In another embodiment of the IL-1 antagonist useful in the method of the invention, examples of anti-IL-1 antibodies are disclosed in U.S. Pat. No. 4,935,343; U.S. Pat. No. 5,681,933; WO 95/01997; EP 0267611, U.S. Pat. No. 6,419,944; WO 02/16436 and WO 01/53353. The IL-1 antagonist of the invention may include an antibody or antibody fragment specific for an IL-1 ligand (e.g., IL-1α or IL-1β) and/or an IL-1 receptor (e.g., IL-1 R1 and/or IL-1 RAcp). Antibody fragments include any fragment having the required target specificity, e.g. antibody fragments either produced by the modification of whole antibodies (e.g. enzymatic digestion), or those synthesized de novo using recombinant DNA methodologies (scFv, single domain antibodies or dAbs, single variable domain antibodies) or those identified using human phase display libraries (see, for example, McCafferty et al. (1990) Nature 348:552-554). Alternatively, antibodies can be isolated from mice producing human or human-mouse chimeric antibodies using standard immunization and antibody isolation methods, including but not limited to making hybridomas, or using B cell screening technologies, such as SLAM. Immunoglobulin binding domains also include, but are not limited to, the variable regions of the heavy (V_{H}) or the light (V_{L}) chains of immunoglobulins.

The term “antibody” as used herein refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant regions, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. Within each IgG class, there are different isotypes (eg. IgG₁, IgG₂, IgG₃, IgG₄). Typically, the antigen-binding region of an antibody will be the most critical in determining specificity and affinity of binding.

An exemplary immunoglobulin (antibody) structural unit comprises a tetramer. Each tetramer is composed of two identical pairs of polypeptide chains, each pair having one light chain (about 25 kD) and one heavy chain (about 50-70 kD). The N-terminus of each chain defines a variable region of about 100-110 or more amino acids primarily responsible for antigen recognition. The terms “variable light chain” (V_{L}) and variable heavy chain (V_{H}) refer to these light and heavy chains respectively.

Antibodies exist as intact immunoglobulins, or as a number of well-characterized fragments produced by digestion with various peptidases. For example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region. While various antibody fragments are defined in terms of the digestion of an intact antibody, one of skill will appreciate that such fragments may be synthesized de novo either chemically or by using recombinant DNA methodology.

Methods for preparing antibodies are known to the art. See, for example, Kohler & Milstein (1975) Nature 256:495-497; Harlow & Lane (1988) Antibodies: a Laboratory Manual, Cold Spring Harbor Lab., Cold Spring Harbor, N.Y.). The genes encoding the heavy and light chains of an antibody of interest can be cloned from a cell, e.g., the genes encoding a monoclonal antibody can be cloned from a hybridoma and used to produce a recombinant monoclonal antibody. Monoclonal antibodies can be humanized using standard cloning of the CDR regions into a human scaffold. Gene libraries encoding human heavy and light chains of monoclonal antibodies can also be made from hybridoma or plasma cells. Random combinations of the heavy and light chain gene products generate a large pool of antibodies with different antigenic specificity. Techniques for the production of single chain antibodies or recombinant antibodies (U.S. Pat. No. 4,946,778; U.S. Pat. No. 4,816,567) can be adapted to produce antibodies used in the fusion proteins and methods of the instant invention. Also, transgenic mice, or other organisms such as other mammals, may be used to express human, human-mouse chimeric, or humanized antibodies. Alternatively, phage display technology can be used to identify human antibodies and heteromeric Fab fragments that specifically bind to selected antigens.

Antibody Screening and Selection

Screening and selection of preferred antibodies can be conducted by a variety of methods known to the art. Initial screening for the presence of monoclonal antibodies specific to a target antigen may be conducted through the use of ELISA-based methods, for example. A secondary screen is preferably conducted to identify and select a desired monoclonal antibody for use in construction of the multi-specific fusion proteins of the invention. Secondary screening may be conducted with any suitable method known to the art. One preferred method, termed “Biosensor Modification-Assisted Profiling” (“BiaMAP”) is described in co-pending U.S. Ser. No. 60/423,017 filed 1 Nov. 2002, herein specifically incorporated by reference in its entirety. BiaMAP allows rapid identification of hybridoma clones producing monoclonal antibodies with desired characteristics. More specifically, monoclonal antibodies are sorted into distinct epitope-related groups based on evaluation of antibody:antigen interactions. Antibodies capable of blocking either a ligand or a receptor may be identified by a cell based assay, such as a luciferase assay utilizing a luciferase gene under the control of an NFKB driven promoter. Stimulation of the IL-1 receptors by IL-1 ligands leads to a signal through NFKB thus increasing luciferase levels in the cell. Blocking antibodies are identified as those antibodies that blocked IL-1 induction of luciferase activity.

Methods of Administration

The invention provides methods of treatment comprising administering to a subject an effective amount of an active agent of the invention. In a preferred aspect, the active agent is substantially purified (e.g., substantially free from substances that limit its effect or produce undesired side-effects). The subject is preferably an animal, e.g., such as cows, pigs, horses, chickens, cats, dogs, etc., and is preferably a mammal, and most preferably human.

Various delivery systems are known and can be used to administer an active agent of the invention, e.g., delivery systems suitable for topical administration, preferably topical administration directly to the eye, or subconjunctival administration, as well as other delivery systems such as those that utilize encapsulation in liposomes, microparticles, microcapsules, recombinant cells capable of expressing the compound, receptor-mediated endocytosis (see, e.g., Wu and Wu,(1987) J. Biol. Chem. 262:4429-4432), construction of a nucleic acid as part of a retroviral or other vector, etc. Methods of introduction are preferably topical or subconjunctival, but may be or enteral or parenteral including but are not limited to intradermal, intramuscular, intraperitoneal, intravenous, subcutaneous, intranasal, and oral routes. The active agents may be administered by any convenient route, for example by absorption through epithelial (e.g. topical administration to the eye) or mucocutaneous linings (e.g., oral mucosa, intestinal mucosa, etc.) or infusion or bolus injection, and may be administered together with other biologically active agents. Administration can be systemic or local. Administration can be acute or chronic (e.g. daily, weekly, monthly, etc.) or in combination or alteration with other agents. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved, for example, and not by way of limitation, by topical administration, subconjunctival administration, local infusion during surgery, e.g., by injection, by means of a catheter, or by means of an implant, said implant being of a porous, non-porous, or gelatinous material, including membranes, such as sialastic membranes, fibers, or commercial skin substitutes.

In another embodiment, the active agent can be delivered in a vesicle, in particular a liposome (see Langer (1990) Science 249:1527-1533). In yet another embodiment, the active agent can be delivered in a controlled release system. In one embodiment, a pump may be used (see Langer (1990) supra). In another embodiment, polymeric materials can be used (see Howard et al. (1989) J. Neurosurg. 71:105). In another embodiment where the active agent of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see, for example, U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

Pharmaceutical Compositions

The present invention also provides pharmaceutical compositions. Such compositions comprise a therapeutically effective amount of one or more active agents, and a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin.

In a preferred embodiment, the composition is formulated in accordance with routine procedures as a pharmaceutical composition adapted for intravenous administration to human beings. Where necessary, the composition may also include a solubilizing agent and a local anesthetic such as lidocaine to ease pain at the site of the injection. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The active agents of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the active agent of the invention that will be effective in the treatment of corneal transplant rejection can be determined by standard clinical techniques based on the present description. In addition, in vitro assays may optionally be employed to help identify optimal dosage ranges. The precise dose to be employed in the formulation will also depend on the route of administration, and the seriousness of the condition, and should be decided according to the judgment of the practitioner and each subject's circumstances. However, suitable dosage ranges for intravenous administration are generally about 20-5000 micrograms of active compound per kilogram body weight. Effective doses may be extrapolated from dose-response curves derived from in vitro or animal model test systems.

A composition useful in practicing the methods of the invention can be a liquid wherein the active agent, for example, the IL-1 trap, is present in solution, in suspension, or both. The term “solution/suspension” as used herein refers to a liquid composition wherein a first portion of the active agent is present in solution and a second portion of the active agent is present in particulate form, in suspension in a liquid matrix. A liquid composition also includes a gel. In a preferred embodiment, the liquid composition is aqueous. Alternatively, the composition can take the form of an ointment. In yet another alternative, the composition can take the form of a solid article that can be inserted in the eye, such as for example between the eye and eyelid or in the conjunctival sac, where it releases the active agent. Release from such an article is usually to the cornea, either via the lacrimal fluid, or directly to the cornea itself, with which the solid article is generally in direct contact. Solid articles suitable for implantation in the eye are generally composed primarily of polymers which can be either bioerodible or non-bioerodible.

The composition is an aqueous solution, suspension or solution/suspension, can be in the form of eye drops. A desired dosage of the active agent can be measured by administration of a known number of drops into the eye. For example, for a drop volume of 25 μl, administration of 1-6 drops will deliver 25-150 μl of the composition. Preferably no more than 3 drops, more preferably no more than 2 drops, and most preferably no more than 1 drop, should contain the desired dose of the active agent for administration to an eye. Administration of a larger volume could result in a loss of a significant amount of the applied composition by lacrimal drainage.

An aqueous suspension or solution/suspension useful for practicing the methods of the invention may contain one or more polymers as suspending agents. Useful polymers include water-soluble polymers such as cellulosic polymers and water-insoluble polymers such as cross-linked carboxyl-containing polymers.

The aqueous suspension or solution/suspension of the present invention is preferably viscous or muco-adhesive, or even more preferably, both viscous or mucoadhesive.

In another embodiment, the composition useful in practicing the methods of the invention is an in situ gellable aqueous composition. Such a composition comprises a gelling agent in a concentration effective to promote gelling upon contact with the eye or with lacrimal fluid. Suitable gelling agents include but are not limited to thermosetting polymers. The term “in situ gellable” as used herein is includes not only liquids of low viscosity that form gels upon contact with the eye or with lacrimal fluid, but also includes more viscous liquids such as semi-fluid and thixotropic gels that exhibit substantially increased viscosity or gel stiffness upon administration to the eye. Skilled artisans will recognize that it can be advantageous to formulate a composition useful for practicing the methods of the invention as a gel to minimize loss of the composition immediately upon administration, generally as a result for example of lacrimation. Although it is desirable that such a composition exhibits a further increase in viscosity or gel stiffness upon administration, this is not required if the initial gel is sufficiently resistant to dissipation by lacrimal drainage.

Further more, aqueous compositions useful for practicing the methods of the invention have ophthalmically compatible pH and osmolality. One or more ophthalmically acceptable pH adjusting agents and/or buffering agents can be included in a composition of the invention, including acids such as acetic, boric, citric, lactic, phosphoric and hydrochloric acids; bases such as sodium hydroxide, sodium phosphate, sodium borate, sodium citrate, sodium acetate, and sodium lactate; and buffers such as citrate/dextrose, sodium bicarbonate and ammonium chloride. Such acids, bases, and buffers are included in an amount required to maintain pH of the composition in an ophthalmically acceptable range. One or more ophthalmically acceptable salts can be included in the composition in an amount sufficient to bring osmolality of the composition into an ophthalmically acceptable range. Such salts include those having sodium, potassium or ammonium cations and chloride, citrate, ascorbate, borate, phosphate, bicarbonate, sulfate, thiosulfate or bisulfite anions.

For systemic administration, a therapeutically effective dose can be estimated initially from in vitro assays. For example, a dose can be formulated in animal models to achieve a circulating concentration range that includes the IC₅₀ as determined in cell culture. Such information can be used to more accurately determine useful doses in humans. Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the compounds that are sufficient to maintain therapeutic effect. In cases of local administration or selective uptake, the effective local concentration of the compounds may not be related to plasma concentration. One having skill in the art will be able to optimize therapeutically effective local dosages without undue experimentation.

The amount of compound administered will, of course, be dependent on the subject being treated, on the subject's weight, the severity of the affliction, the manner of administration, and the judgment of the prescribing physician. The therapy may be repeated intermittently while symptoms are detectable or even when they are not detectable. The therapy may be provided alone or in combination with other drugs.

Standard methods for assessing corneal inflammation and neovascularization can be used to determine whether a subject is positively responding to treatment with the IL-1 blockers. Generally the physician will monitor the subject at periodic points post-injury or post-surgery to assess whether the subject is benefiting from administration of the IL-1 blocker. In the case of corneal transplant, such post-surgery assessment will include tests to identify epithelial rejection which is defined as the formation of an epithelial line appearing as a raised ridge of epithelium; subepithelial rejection which is defined as subepithelial infiltrates that resemble those seen in epidemic keratoconjunctivitis; stromal rejection which is defined as stromal infiltrates that progress towards the center of the graft; and endothelial rejection which is diagnosed when one or more of the following features are present: Khodadoust line, keratic precipitates, stromal edema, or aqueous cells (Naacke, HG, et al., (2001) Cornea 20(4):350-353). Thus, these as well as other methods known to the art may be used to determine the extent to which the methods of the present invention are effective at treating, preventing or reducing the incidence of corneal inflammation and neovascularization.

Combination Therapies

In numerous embodiments, the IL-1 blockers of the present invention may be administered in combination with one or more additional compounds or therapies or medical procedures. For example, suitable therapeutic agents for use in combination, either alternating or simultaneously, with the IL-1 blockers may include topically administered immunosuppressive agents such as corticosteroids, dexamethasone, cyclosporin A, or anti-metabolic agents or systemically administered immunosuppressive agents such as corticosteroids, dexamethasone, cyclosporin A, FK506, or anti-metabolic agents, as well as other agents effective to treat, reduce, or prevent corneal inflammation and neovascularization associated with corneal injury, including corneal surgery such as corneal transplant (see Barker et al. (2000) Clin Exp Opthal 28:357-360).

For example, a suitable therapeutic agent for use in combination, either alternating or simultaneously, with the IL-1 antagonists may include anti-platelet therapy such as aspirin, Reopro™ (Lilly), anti-p-selectin antibodies; antithrombolic and blood thinning agents, such as Retavse™ (Centocor); Streptase™ (AstraZeneca), TNKase™ (Genentech), Ticlid™ (Roche) and Plavix™ (Bristol-Myers Squibb) and heparin; HMG-CoA reductase inhibitors, such as Baycol™ (Bayer), Lescol™ (Noavartis), Lipitor™ (Pfizer), Mevacor™ (Merck), Pravachol™ (Bristol Myers Squibb, Zocor™ (Merck) or antilipidemic agents such as, Colestid™ (Pfizer), WelChol™ (Sankyo), Atromid-S™ (Wyeth), Lopid™ (Pfizer), Tricor™ (Abbott); agents effective to treat or prevent restenosis such as Sirolimus™ (Wyeth, Johnson & Johnson), dexamethasone (Merck), Predisolone™ (Muro, Mylan, Watson, We), Tacrolimus™ (Fujisawa), Pimecrolimus™ (Novartis) Taxol/Paclitaxel (Bristol-Myers Squibb), or Methotrexate (Baxter, Mylan, Roxane); anti-fibrolytic agents such as antibodies against TGFβ PDGF, or CTGF; PDGF inhibitors such as Gleevec™ (Novartis); anti-inflammatory agents such as antibodies, peptides and other inhibitors of CD11 a/CD8 (Mac1) [Raptiva™ (Genentech)], ICAM, C5a and TNFα [Humira™ (Abbott), Enbrel™ (Amgen), Remicade™ (Centocor)], Thalidomide™ (Celltech); hypertension drugs, such as ACE inhibitors [Accupril™ (Parke-Davis); Altace™ (Monarch); Captopril™ (Mylan); Enalaprilate™ (Baxter); Lotensin™ (Novartis); Mavik™ (Bristol-Myers Squibb); Prinivil™ (Merck); Univasc™ (Schwarz), Vasotec™ (Merck)]. In addition the IL-1 antagonists may be used in combination, either alternating or simultaneously, with surgical procedures including but not limited to surgical stenting and balloon angioplasty.

Kits

The invention also provides an article of manufacturing comprising packaging material and a pharmaceutical agent contained within the packaging material, wherein the pharmaceutical agent comprises at least one IL-1-specific fusion protein of the invention and wherein the packaging material comprises a label or package insert which indicates that the IL-1-specific fusion protein can be used for treating corneal inflammation and neovascularization.

### Specific Embodiments

Using an animal model of corneal injury, the ability of an IL-1 trap antagonist to reduce, prevent or treat IL-1-mediated inflammation and neovascularization occurring after corneal injury was investigated. As shown below, administration of an IL-1 receptor-based blocker of IL-1-mediated activity was able to prevent IL-1-mediated inflammation and neovascularization in this animal model.

Other features of the invention will become apparent in the course of the following descriptions of exemplary embodiments which are given for illustration of the invention and are not intended to be limiting thereof.

### EXAMPLES

The following example is put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Inhibition of Il-1 Blocks Pathological Corneal Angiogenesis and Associated Inflammation

Materials and Methods: Male C57BL/6 mice were used in this experiment (n=30). Corneal inflammation and neovascularization were induced by intrastromal placement of 3 nylon sutures in the right eye. Twenty-four hours before corneal injury, C57BU6 mice were injected intravenously with either Ad.IL-1 trap at 1×10A9 pfu (a chimeric protein comprising the extracellular, ligand binding domains of murine IL-1 R1 and murine IL-1 RAcP and the Fc domain of mouse immunoglobulin) or a control adenovirus (adenovirus Fc). The growth of corneal neovessels was evaluated on days 8 and 15 by slit-lamp microscopy and histology. The vasculature was labeled with an endothelial-specific fluorescein-conjugated lectin (lycopersicon esculentum) and neovascularization was evaluated in corneal flat-mount. The Scion Image program was used for analysis of the area and length of corneal neovessels. Circulating levels of murine IL-1 trap were measured by ELISA 2, 10, and 15 days after delivery of IL-1 trap.

The intrastromal placement of nylon sutures into the cornea provokes a foreign-body response characterized by local inflammation and neovascularization. A single systemic injection of Ad.IL-1 Trap significantly inhibited the corneal neovascularization (96% reduction), compared with PBS treated or Ad.Fc treated animals. Applicants also found that Ad.IL-1 trap decreased the infiltration of inflammatory cells into the injured cornea. In contrast, the structure and function of the normal limbal corneal vasculature was unaffected in the contralateral, undamaged eye.

1. A method of treating or inhibiting corneal inflammation and/or corneal neovascularization in a mammal, comprising administering to the mammal an interleukin-1 (IL-1) antagonist.

2. The method of claim 1, wherein the corneal inflammation and/or corneal neovascularization results from corneal transplant surgery.

3. The method of claim 1, wherein the IL-1 antagonist blocks IL-1 activity or expression.

4. The method of claim 3, wherein the IL-1 antagonist is selected from the group consisting of an anti-IL-1 antibody or antibody fragment, an anti-IL-1 R1 antibody or antibody fragment, an antilL-1 RAcp antibody or antibody fragment, an IL-1 trap, IL-1 Ra, an antisense molecule, an inhibitory ribozyme designed to catalytically cleave gene mRNA transcripts encoding IL-1α, IL-1 β, IL-1 R1, IL-1RAcp, and a short interfering RNA (siRNA) molecule.

5. The method of claim 4, wherein the IL-1 trap comprises (i) one or more IL-1 receptor components or fragments thereof, (ii) one or more antibody or antibody fragments specific to an IL-1 ligand or an IL-1 receptor, or fragments thereof, or a combination of receptor components and antibody fragments, and (iii) a multimerizing component.

6. The method of claim 5, wherein the multimerizing component is an immunoglobulin-derived domain.

7. The method of claim 6, wherein the IL-1 trap comprises the amino acid sequence of SE ID NO:2, 4, 6, 8, 10, 12, 14, 16, 18, 20, 22, 24, or 26.

8. The method of claim 7, wherein the IL-1 trap comprises the amino acid sequence of SEQ ID NO:1.

9. The method of claim 1, wherein the mammal is a human.

10. The method of claim 1, wherein the administration is subcutaneous, intramuscular, intranasal, intraarterial, intravenous, topical, or suconjunctival.

11. A method of reducing or ameliorating the incidence of corneal inflammation and/or corneal neovascularization in a subject in need or at risk for development thereof, comprising administering an interleukin-1 (IL-1) blocker or antagonist such that corneal inflammation and/or corneal neovascularization is reduced or ameliorated.

12. An article of manufacturing, comprising:
(a) packaging material; and
(b) a pharmaceutical gent contained within the packaging material;
wherein the pharmaceutical agent comprises at least one interleuking-1 (IL-1) trap of the invention and wherein the packaging material comprises a label or package insert which indicates the IL-1 trap can be used for the treatment of corneal inflammation and/or corneal neovascularization.
